(19) United States
(12) Patent Application Publication
(10) Pub. No.: US 2005/0147960 A1
(43) Pub. Date: Jul. 7, 2005

(54) In vitro development of tissues and organs

(76) Inventors: Douglas Levinson, Sherborn, MA (US); Robert S. Langer, Newton, MA (US); John Lucas, San Diego, CA (US)

Correspondence Address: TRANSFORM PHARMACEUTICALS, INC., 29 HARTWELL AVENUE, LEXINGTON, MA, 02421, US

(21) Appl. No.: 10/960,743

(22) Filed: Oct. 7, 2004

Related U.S. Application Data
(60) Provisional application No. 60/510,808, filed on Oct. 10, 2003.

(51) Int. Cl. C12Q 1/00; C12N 5/08

(52) U.S. Cl. 435/4; 435/366; 435/404

(57) ABSTRACT

A method for producing a differentiated tissue is provided, which method comprises isolating at least one population of homogeneous multipotent cells, and culturing the cells in the presence of at least one differentiation factor, wherein the cells are supported by a solid phase scaffold. Differentiated tissues produced by this method, and there medical uses are also described. A method of screening for a process of producing a given differentiated tissue, and a method of optimizing a process of producing a given differentiated tissue are also described.

### CROSS-REFERENCE TO RELATED APPLICATION

The present application claims the benefit of priority of U.S. Provisional Application Ser. No. 60/510,808, filed Oct. 10, 2003, which is hereby incorporated by reference herein in its entirety, including any figures, tables or drawings.

### FIELD OF THE INVENTION

This invention is directed to methods for producing differentiated tissues, differentiated tissues obtained thereby, and solid phase scaffolds therefore.

### BACKGROUND OF THE INVENTION

When cultured in the presence of leukemia inhibitory factor or appropriate feeder cell layers, mammalian embryonic stem cells are capable of dividing indefinitely without entering into any developmental pathway or differentiating. Removal of leukocyte inhibitory factor and/or appropriate feeder layers from the culture medium results in differentiation of the embryonic stem cells. Embryonic stem cells are capable of differentiating into all cell types and are described as pluripotent.

The observation that embryonic stem cells can differentiate into mature cell types in vitro has suggested to many that it may be possible to develop complex tissues in vitro for grafting and transplantation purposes. However, to date, no method for consistently producing complex structured human tissues and organs in vitro is available. It is hypothesized that this failure is due, in part, to the inability to effectively recapitulate in vitro, the complex inductive microenvironments that normally give rise to complex 3 dimensional tissues and organs in vivo. Methods are therefore needed to 1) identify essential individual microenvironmental features and 2) identify how those essential features need be combined both spatially and temporally to support/promote complex tissue development in vitro.

In the normal course of development of a mammal, embryonic cells gradually lose the ability to form particular tissues in a manner that is dependent upon its position in the body. This is thought to result from the particular combination of signals received at certain points during the development by the cell, and the intensity of these signals. The combination and intensity of signals received by responding cells within a spatially defined anatomical region constitutes the microenvironment of the cell. An inductive microenvironment is an environment that permits or promotes a change in a developmental, differentiative, proliferative state in at least some of the participating cell types. Further, certain features of the microenvironment are expected to change through time and it is recognized that the correct temporal succession of changes of microenvironmental features are important to the development of complex tissues and organs and cellular differentiation. These temporally related microenvironments may share one or more features in common with each other. Herein, differentiation is defined as a change of cellular state that is consistent with progression from an earlier precursor stage to a later more mature stage. De-differentiation is the reverse of that process in which a cell of a more mature stage transitions to less mature stage.

Herein, development is referred to as a biological process in which the body of a multicellular organism or constituent parts of a body of a multicellular organism (including tissues, organs, and glands) progress from a less mature state to a more mature state in development. The development of organs and complex structured tissue can be referred to as organogenesis.

In vivo development can be delineated into distinct stages including early embryonic development, morphogenesis, pattern formation, organogenesis.

The development of tissues and organs is a highly complex and ordered process which requires specific signals to be provided to cells at particular times in the developmental process. The level of the signal received by a cell may also be critical. Current attempts to generate organs and tissues in vitro have failed because they do not take account of all these factors. A need thus exists for a method of developing tissues and organs in vitro which mimics the natural microenvironment surrounding participating cells during development.

### SUMMARY OF THE INVENTION

Accordingly, in a first aspect, the present invention provides a method for the discovery of microenvironmental parameters that result in producing a differentiated tissue, which method comprises isolating at least one population of homogeneous multipotent cells, and culturing the cells on a solid phase scaffolding the absence or presence of at least one differentiation factor

As used herein, a “differentiated tissue” is a population of cells that are spatially organized in the same manner as found in a developing or mature multicellular organism, e.g., animal or human. In an embodiment the differentiated tissue comprises at least 1, 2, 3, 4 or more different cell types. Microenvironmental parameters include, structural support or scaffold, the populations of cells, the composition of liquid media including soluble agents that are capable of causing a cellular resonse, the matrix material present on the scaffold, electrical stimulation, mechanical stimulation, change in temperature or pressure, the change of any of these parameters through time. In the context of the present invention, a multipotent cell is defined as a cell whose fate has not been determined (i.e. it is capable of differentiating into more than one cell type), but which is not unrestricted in the cell types it may form (i.e. unlike a pluripotent cell, a multipotent cell is able to form only a subset of all possible cell types). A homogeneous population of multipotent cells is defined as a collection of cells that exhibit a common feature (e.g., expression of a marker useful in detection). Homogenous populations of multipotent cells may contain cells that have identical, similar or different developmental potentials. The solid, or gel, phase scaffold may be any substrate capable of physically supporting cells within a 3-D structure. There are three general types of scaffold structures. These include structural scaffolds with an imposed pore structure, gel-type scaffolds formed in situ in the presence of cells or tissues; natural tissue derived gels. A key requirement is an interconnected porosity of larger dimensions than resident cells. Woven and non-woven fiber based fabrics have been developed. Shapes may be formed by using freeze drying, particulate leaching, foaming and solid free form fabrication, and 3-D printing.

The precise three-dimensional shape of the substrate is not particularly limited. However, in a preferred embodiment, the scaffold is in the form of a mesh or network of fibres, having pores or interstices of a dimension sufficient to enable cells to be supported in the interior of the scaffold. The dimensions of pores are typically 20 μm or lower. More preferably, the dimensions of pores are 10 μm or lower, and even more preferably, the dimensions of the pores are 1 μm or lower.

The scaffold typically supports the multipotent cells. The term “supports” refers to the situation where cells are adherent to the interior and exterior surfaces of the scaffold, and also the situation where the cells are simply resting on the interior and exterior surfaces of the scaffold. Typically, substantially all of the population of multipotent cells will adhere to the surfaces of the scaffold. However, cells undergoing division do not adhere to the surfaces of the scaffold and may simply rest in position. Different scaffold materials may differ in mechanical properties, stabilities (and may be biodegradable), ability to assume a macroscopic shape (e.g., sheet vs spherical), and biocompatibility. Scaffolds may be synthetic, naturally derived or semisynthetic. Polymers include degradable synthetic bulk polymers. These include: 1) synthetic polyesters (including polyesters derived from lactide, glycolide, and caprolactoine). Other examples include poly hydroxybutyrate, coplymers of polyhydroxybutyrate with hydroxyvalerate, poly-4 hydroxyputyrate, 2) Synthetic gels (including PEO based substrates), 3) Natural polymers derived from extracellular matrix proteins and derivatives (e.g., collagen) and materials derived from plants and seaweed. Members of this group include type I collagen, laminin family proteins and fibrin. Matrices may be derived from by extraction or partial purification of whole tissue and may contain residual growth factors. Natural polysaccharides are another class which includes hyaluonic acid, alginate. 4) Synthetic materials with tailored biological ligands (examples include the inclusion of fibronectin RGD adhesion recognition sequences and other peptides that promote cellular adhesion, and cellular growth factors and nutrients).

Scaffold materials may be derived from mixtures of more than one material and may be associated with one or more elements that make up a matrix. Examples of a suitable scaffold material include poly(propylene fumarate-co-ethylene glycol), poly(caprolactone), poly(ethylene oxide)poly-4-hydroxybutyrate, poly lactide co glycolide(PLG), extracellular matrix derived from natural sources, gelatin, collagen, fibrinogen, hyaluronic acid.

The differentiation factor comprises any factor capable of promoting a multipotent cell to differentiate. Differentiation factors may be identified by culturing multipotent cells in the presence of putative factors and identifying a differentiation factor as a factor which causes at least some of the cells to differentiate. Preferably the factor will cause 10% of the population of multipotent cells to differentiate to form any given cell type. More preferably, at least 20%, 30%, 40%, 50% 60%, 70%, 80% or at least 90% of the population of multipotent cells differentiate to form the given cell type. Methods of identifying differentiated cells are described below. Differentiation factor may be added to the cultures or generated by the cells contained within the cultures. Examples of differentiation factors include secreted proteins (such as growth factors, morphogens, cytkines, chemokines), proteoglycans, carbohydrates, small drug like molecules, metabolites, and nutrients.

Differentiation factors can be used to create multiple different microenvironments within the same matrix. The positioning of differentiation factors on a scaffolding or within a matrix can affect the concentration or the timing for which a factor can interact with a specific cell or population of cells. In one embodiment, differentiation factors are positioned in different positions on a scaffolding. By positioning factors on different positions, the factor can leech into a matrix. Thus, in some instances, the factor concentration will be highest closer to the factor position and lowest farther from the factor position. If more than one factor is used and more than one position is used, gradients of factor concentration can be created. For example, if Factor A is positioned on one side of a matrix and Factor B is positioned on another side of a matrix, then the matrix will consist of varying microenvironments with differing concentrations of Factor A and Factor B. The number of factors used is not limited and can include 2, 3, 4, 5, 6, or more factors in 2, 3, 4, 5, 6, or more different positions. Factors can enter a matrix at varying speeds. One factor may enter the matrix quickly while another factor may enter the matrix slowly. Changing the speed of factor entry from the factor position into the matrix can alter the microenvironment. In one embodiment, a scaffolding is set up on the format of a box and each corner of the box contains a different factor. In this embodiment, factor can move, leech, or spread from each corner to create multiple microenvironments with different combinations of factor. These different combinations of factor can be used to induce or detect changes to a cell or cell population.

Differentiated tissues obtained by the method of the invention are also provided. These differentiated cells may be used in medicine. In one embodiment, differentiated tissues produced by the method of the invention may be used for transplantation into a patient in need thereof. Additionally, microenvironments discovered by this approach may also be used in medical applications. Both microenvironments and derived tissues obtained by this approach may have utility in predicting physiological responses to challenge with test conditions. For example, microenvironments may have utility in testing the teratogenicity of test compounds or environmental agents. Tissues obtained in this approach may have further utility as a basis for high level production of cells and cell derived molecules (e.g., proteins) that may have therapeutic utility or non-therapeutic utility. Tissues obtained in this approach may have further utility as biosensors to detect the presence of test agents.

In a further aspect, the invention provides a screening method for identifing a process for producing a given differentiated tissue. The screening method comprises providing a plurality of test populations of multipotent cells, wherein each test population of multipotent cells comprises either an isolated population of homogeneousmultipotent cells, or a mixture of at least two populations of isolated homogeneous multipotent cells, culturing each test population of multipotent cells on a solid phase scaffold in the presence or absence of at least one differentiation factor (not necessary-biologically speaking-could be no added factor present), assaying each cultured test population for the presence of a differentiated cell type present in the differentiated tissue, and/or possessing a 3-dimensional structural organization and identifying a process for reliably producing a given differentiated tissue as a process that generates a differentiated cell type present in the differentiated tissue. In yet another aspect, the invention provides a method of optimizing a process for producing a given differentiated tissue through controlled development or self-assembly. Herein, controlled development refers to the ability of cells within a microenvironment to give rise to multicellular structures possessing qualities of a biological tissue through a process of continued development and differentiation. Herein, self assembly refers to the ability of cells, when recombined under appropriate conditions to organize into multicellular structures that posses biological properties of a tissue. The method of optimizing a process for producing a given differentiated tissue involves modifying at least one parameter of a known process for producing the differentiated tissue, thereby arriving at a modified process, performing the modified process, determining the percentage of cells that have differentiated to form a differentiated cell type present in the differentiated tissue, and/or structurally organized in a tissue like pattern (e.g., sheets, tubes, and branching structures) and identifying an optimized process as a modified process which results in a higher percentage of differentiated cells than the known process or percentage higher order structure. Optionally, the steps of modifying the process and testing the modified process are repeated a number of times to provide an optimized process.

In this context, the term parameter refers to any variable factor in the process of producing a differentiated tissue. Parameters include the scaffolding material, the shape of the scaffold, the composition of the matrix deposited on the scaffold, the composition of cells, the presence of factors in solution or on the matrix or scaffold, temperature, pressure, physical stimulation (including mechanical and electrical and optical), exposure to gas (such as air), and the temporal order in which any of the parameters are manipulated.

In a further aspect, the invention provides a solid phase scaffold for culturing cells, which is associated with at least one differentiation factor (not necessarily).

The differentiation factor may be bound to an exterior or interior surface of the solid phase scaffold. Alternatively, the differentiation factor may simply be encapsulated or entrapped within the solid phase scaffold, without being physically bound to any surface of the scaffold.

### DETAILED DESCRIPTION OF THE INVENTION

The invention will now be described in greater detail, by way of example only.

Isolation of Homogeneous Populations of Multinotent Cells

The population of multipotent cells homogeneous for at least one feature, e.g., a biological marker, may be derived from cultured embryonic stem cells. Any established embryonic stem cell line may be used in this invention. Additionally, primary stem cell populations may be used that are derived from embryos of mammals by isolation of inner cell mass. Additionally, stem cells may be derived from nuclear transfer (for example into enucleated oocytes). Preferably, the embryonic stem cell lines (and thus the multipotent cells) are derived from a primate or a rodent. Human or mouse embryonic stem cell lines are particularly preferred.

Embryonic stem cells may be cultured in vitro using techniques known in the art (for reviews see Robertson E. J. Ed., Oxford IRL Press, Teratocarcinomas and Embryonic Stem Cells; A Practical Approach, 1987, and Hogan et al., Cold Spring Harbour Laboratory Press, Manipulating the Mouse Embryo 2^{nd} Ed., 1994). Typically, embryonic stem cells are cultured on a mitotically inactive feeder cell layer in a D-MBM formulation supplemented with Foetal Bovine Serum or KNOCKOUT™SR (Invitrogen Corporation, 1600 Faraday Avenue, PO Box 6482, Carlsbad, Calif. 92008), L-glutamine, non-essential amino acids, β-mercaptoethanol and antibiotics. Additionally, leukaemia inhibitory factor or basic fibroblast growth factor (bFGF) may be required to prevent differentiation of the embryonic stem cells. Optimal culture media have been identified for established embryonic stem cell lines.

The H9 human embryonic stem cell line is particularly preferred. A method for culturing this cell line is described in Itskovitz-Eldor et al. (Mol. Med. 6: 88-95, 2000).
BresaGen, Inc., Athens, Ga.
Cell lines: hESBGN.01, hESBGN.02
CyThera, Inc., San Diego. Calif.
Cell lines: hES-1-2, hES-3-0, hES4-0, hES-5-1, hES-8-1, hES-8-2, hES-9-1, hES-9-2, hES 10
ES Cell International. Melbourne. Australia Cell lines:
Cell lines HES-1, HES-2, HES-3, HES-4, HES-5, HES-6
Geron Corporation, Menlo Park, Calif.
Cell lines: H1, H7, H9, H13, H14, H9.1, H9.2
Göteborg University, Göteborg, Sweden
Cell lines: Salgrenska-1, Salgrenska-2, Salgrenska-3,
Karolinska Institute, Stockholm. Sweden
Cell lines: hlcm8, hlcm9, hlcm40, hlcm41, hlcm42, hlcm43,
Maria Biotech Co. Ltd.—Maria Infertility Hospital Medical Institute, Seoul, Korea
Cell lines: MB01, MB02, MB03
MizMedi Hospital—Seoul National University, Seoul, Korea
Cell lines: Miz-hES-1
National Centre for Biological Sciences/Tata Institute of Fundamental Research, Bangalore, India
Cell lines: FCNCBS1, FCNCBS2, FCNCBS3
Pochon CHA University, Seoul, Korea
Cell lines: CHA hES-1, CHA hES-2
Reliance Life Sciences, Mumbai, India
Cell lines: RLS ES 5, RLS ES 7, RLS ES 10, RLS ES 13, RLS ES 15, RLS ES 20, RLS ES 21
Technion University. Haifa, Israel
Cell lines: I3, I3.2, I3.3, I4,I6, I6.2, J3, J3.2
University of California, San Francisco, Calif.
Cell lines: HSF4
Wisconsin Alumni Research Foundation, Madison. Wis.
Cell lines: H1, H7, H9

The embryonic stem cells may contain at least one reporter gene, each of which is under the control of a different tissue specific promoter. The reporter gene may be present upon a DNA construct or may be integrated into the genome. It is preferred however, that the reporter gene is stably heritable, and must be reliably passed down to daughter cells following cell division.

The introduction of the reporter gene under the control of a tissue specific promoter may be effected by standard molecular biology methods.

In a preferred embodiment, the reporter gene encodes a fluorescent product such as eGFP, eCFP, eYFP and DsRed. In another embodiment, the reporter gene may encode a protein not expressed in any cell of the species in question e.g. a bacterial protein. Preferably, the protein product of the reporter gene is recognized by a commercially available antibody (i.e. a commercially available antibody capable of selectively binding the protein product).

A tissue specific promoter is any promoter that causes transcription of an associated gene in a single cell type or a specific set of cell types. Transcription may further be limited temporally based on environmental conditions (e.g., paracrine, endocrine, or autocrine stimuli) or stage of development. Suitable tissue specific promoters include the myosin heavy chain promoter (specific for cardiomyocyte differentiation), the brachyury promoter (expressed in mesendodermal cells), the Surfactant Protein C (SPC) promoter (specific for lung cells) and the insulin promoter (specific for pancreatic islet cells). The sequences of these promoters are known for both mice and humans.

A large number of developmentally regulated genes have been characterized with respect to expression during embryogenesis or organ development. Examples include T box family members, FGF receptor family members (e.g., FGFR2, FGFR7, FGFR8, FGFR12, FGFR13), GATA binding transcription factors, activin beta-A, bone morphogenic protein family members (e.g., BMP-2,4, 5, and 7), Dll1, Dll4, fas ligand, Follistatin GDNF, HB-GAM, HDGF, HGF, IGF I, IGF II, Jag-1, MidKine, NTN, NT3, PDGF-A, PDGF-B, Pleiotrophin, TGF-alpha, TGF-betal, VEGF, Wnt family members (Wnt 1, 3, 3a, 5a, 12), 14-3-3 epsilon, SET, CaBP9K, HOX family members, PDX family members , hedgehog members (including sonic hedgehog and Indian hedgehog, myocardin, HOP, vasoactive intestinal peptide, galanin, Noggin, Chordin, Sox, DKK-1, DKK-2, HNF-3, SMAD 1, 2, 3, 4, 5, 8, KGF, EGF, insulin, glucagons, pancreatic polypeptide, Reg-1, Reg-2, somatostatin, flk-1, flk2, prox-1, STF-1, tlx-1, eps-8, eps-15, c-Ret, androgen receptor, estrogen receptor, myosin heavy chain, surfactant protein C, albumin. The promoters for some of these genes have been cloned and sequenced.

The population of homogeneous multipotent cells is obtained from the differentiation of an embryonic stem cell line to form a heterogeneous population of multipotent cells, followed by isolating a homogeneous population of multipotent cells from this heterogeneous population.

Differentiation of the embryonic cell line to form a heterogeneous population of multipotent cells is performed by modification of the conditions used to culture the embryonic stem cells. The culture conditions may be modified by removal of a factor required for continued proliferation of embryonic stem cells. In other words, a factor required to maintain embryonic stem cells in an undifferentiated state may be removed. Factors required to maintain embryonic stem cells in an undifferentiated state typically include a feeder cell layer, leukemia inhibitory factor and basic FGF.

Alternatively, the culture conditions can be modified to promote differentiation by the inclusion of a stem cell differentiation factor in the culture medium. A stem cell differentiation factor comprises any factor capable of promoting a pluripotent embryonic stem cell to differentiate into any cell type. Stem cell differentiation factors may be identified by culturing pluripotent embryonic stem cells in the presence of putative factors and identifying a differentiation factor as a factor which causes some, or at least 10% of the population of pluripotent embryonic stem cells to differentiate to form any given cell type. More preferably, at least 20%, 30%, 40%, 50% 60%, 70%, 80% or at least 90% of the population of pluripotent embryonic stem cells differentiate to form the given cell type. Some stem cell differentiation factors may also trigger the differentiation of multipotent cells.

Stem cell differentiation factors include factors known to be involved in early embryogenesis including sonic hedgehog, retinoic acid, members of the TGFβ superfamily of signaling proteins and IGF. Stem cell differentiation factors may also promote particular pathways of development, and include cardiomyocyte promoting factors, lung promoting factors.

Additional factors include:

Bone morphogenic protein family members (e.g., BMP-2,4, 5, and 7), Dll1, Dll4, fas ligand, Follistatin GDNF family members, neurotrophins (e.g., BDNF NT3), HB-GAM, HDGF, HGF, IGF I, IGF II, Jag-1, MidKine, NTN, PDGF-A, PDGF-B, Pleiotrophin, TGF family members, TGF-alpha, TGF-betal, VEGF, Wnt family members (Wnt 1, 3, 3a, 5a, 12), 14-3-3 epsilon, SET, CaBP9K, hedgehog members (including sonic hedgehog and Indian hedgehog, myocardin, HOP, vasoactive intestinal peptide, galanin, Noggin, Chordin, DKK-1, DKK-2, HNF-3, KGF, EGF, insulin, glucagons, pancreatic polypeptide, somatostatin, flk-1, flk2, prox-1, STF-1, tlx-1, eps-8, eps-15, c-Ret, androgen, estrogen, TNF, retinoic acid, syndecans.

The culture conditions can also be modified to promote differentiation by the inclusion of a differentiated cell layer. For example, embryonic stem cells may be stimulated to differentiate to form haematopoietic cell types in the presence of non-mitotic bone marrow stromal cells, such as S17 cells.

Conditions required to trigger differentiation of established cell lines are known. For example, the H9 cell line may be induced to differentiate by treatment of cell colonies with 1 mg/ml collagenase type IV, followed by resuspending the cells in differentiation media without Leukaemia inhibitory factor and basic FGF in petri dishes to induce the formation of embyroid bodies according to the method of Itskovitz-Eldor et al. (Mol. Med. 6: 88-95, 2000).

The resulting population of multipotent cells is heterogeneous i.e. it comprises several different cell types, each of which has a distinct developmental potential. The cells are then suspended in media such that substantially all of the cells do not adhere to each other or to the wall of the culture vessel. Methods for suspending cultured cells are known in the art and include treatment of the cells with trypsin or collagenase. The suspended cells can then be sorted or profiled into groups expressing the same target protein, or same set of target proteins.

This may be achieved by contacting the cells with a profiling protein. A profiling protein is any protein capable of binding specifically protein to a target protein that is expressed in at least one differentiated tissue, which target protein is not expressed in embryonic stem cells. Typically, the profiling protein is an antibody. In this application, the word antibody encompasses polyclonal antibodies, monoclonal antibodies, single chain antibodies, chimeric antibodies, fragments derived from proteolysis of whole antibodies and/or by reduction of disulphide bonds, or antibodies generated by means of expression libraries. The profiling protein is preferably labeled. Tissue specific promotors can also be used to drive expression of markers useful in detecting a population.

Cells bound to the profiling protein may then be separated, from the remaining cells. Thus, using a single profiling protein, two profiles or populations of cells are formed, a first profile consisting substantially of cells containing the target protein, and a second profile consisting substantially of cells lacking the target protein.

Typically, a panel of profiling proteins are employed. This may be used to generate several profiles of cells, each of which consists substantially of cells containing or lacking the same set of proteins. Cells falling within a profile are described as homogeneous since they express the same target proteins. It is hypothesized that cells sharing the same pattern of expression of target proteins will also share the same pattern of expression of other proteins, and thereby have the same developmental potential. The term developmental potential refers to the range of cell types that the cell is capable of forming when cultured in isolation.

The method for separating cells is not limited. In a preferred embodiment, the profiling protein is labeled with a fluorescent marker and flow cytometry is used to separate labeled cells from unlabeled cells. Typically, cells in a suspension traveling in single file are passed through a vibrating nozzle which causes the formation of droplets containing a single cell or none at all. The droplets pass through a laser beam which excites the fluorescent marker to fluoresce. The fluorescence from each individual cell in its droplet is measured by a detector. After the detector the stream of cells in a suspension pass through an electrostatic collar which gives the droplets a surface charge. The cell carrying droplets are given a positive or negative charge. If the drop contains a cell that fluoresces with an intensity above a particular threshold, the drop is given a charge of one polarity. Droplets containing unlabeled cells get a charge of the opposite polarity. The charged droplets are then deflected by an electric field and, depending upon their surface charge, are directed into separate containers and counted. Droplets that contain more than one cell scatter light more than individual cells. This is readily detected and so these are left uncharged and enter a third disposal container.

Multichannel fluorescent detection devices have been constructed that can separate cells on the basis of labeling with multiple different fluorescent labels. These have multiple lasers which can excite fluorescence at different frequencies and the detector will detect different emission frequencies. Such devices can be used to sort cells on the basis of more than one profiling protein simultaneously and are particularly preferred.

Any fluorescent label may be used in conjunction with flow cytometry. Suitable fluorescent labels include green fluorescent protein, yellow fluorescent protein, rhodamine and texas red, FITC (fluorescein isothiocyanate), Cy3 (indocarbocyanine) and Cy5 (indodicarbocyanine). Panning (i.e., coating a bacterial plate with profiling protein and incubint the cells on top, profiling proteins adherent on particles that can then be physically removes such as magnetic beads, complement mediated depletion.

In a further aspect, the population of homogeneous multipotent cells is not obtained by profiling differentiated cells derived from embryonic stem cells. Instead, adult stem cell lines may be used in place of embryonic stem cell lines. The adult stem cells may contain at least one reporter gene, under the control of a tissue specific promoter.

Scaffold Preparation

The solid phase scaffold may comprise any material that is non-toxic to multipotent cells. Preferably, the multipotent cells are capable of adhering to the scaffold material. Suitable scaffold materials include natural extracellular matrix materials such as collagen and matrigel (BD Biosciences, Bedford, Mass.), and synthetic materials such as carbon fibre, calcium phosphate and polymeric materials. Biodegradable polymeric materials are particularly preferred. These include polylactide co-glycolide (Boehringer Ingelheim, Inglehiem, Germany) and polylactide (Polysciences, Warrington, Pa.). Methods for preparing scaffolds are known in the art.

In a preferred embodiment, a scaffold consisting of a 50/50 blend of poly(lactic-co-glycolic) acid and poly(L-lactic acid) is used. This may be prepared by the salt leaching process, which would be well known to one skilled in the art.

Scaffolds which may be used in the present invention are also available commercially. Such scaffolds include PuraMatrix™ and PuraMatrixCST™ (3DM Inc., Cambridge, Mass., USA).

Scaffolds may be coated with extracellular matrix molecules such as fibronectin and collagen.

The scaffold may be associated with at least one differentiation factor. As discussed above, a differentiation factor is any factor capable of inducing differentiation in cultured multipotent cells. Differentiation factors include factors known to be involved in early embryogenesis including sonic hedgehog, retinoic acid, members of the TGFβ superfamily of signaling proteins and IGF. Differentiation factors may also promote particular pathways of development, and include cardiomyocyte promoting factors, lung promoting factors.

Additional factors include:

Bone morphogenic protein family members (e.g., BMP-2,4, 5, and 7), Dll1, Dll4, fas ligand, Follistatin GDNF, HB-GAM, HDGF, HGF, IGF I, IGF II, Jag-1, MidKine, NTN, NT3, PDGF-A, PDGF-B, Pleiotrophin, TGF family members, TGF-alpha, TOF-betal, VEGF, Wnt family members (Wnt 1, 3, 3a, 5a, 12), 14-3-3 epsilon, SET, CaBP9K, hedgehog members (including sonic hedgehog and Indian hedgehog, myocardin, HOP, vasoactive intestinal peptide, galanin, Noggin, Chordin, DKK-1, DKK-2, HNF-3, KGF, EGF, insulin, glucagons, pancreatic polypeptide, somatostatin, flk-1, flk2, prox-1, STF-1, tlx-1, eps-8, eps-15, c-Ret, androgen, estrogen, TNF, retinoic acid, syndecans.

The association of a differentiation factor with the scaffold may result from the differentiation factor being bound to the scaffold material via covalent bonds, hydrogen bonds, hydrophobic interactions or van der Waals forces, or simply by the encapsulation of the differentiation factor in the pores of the scaffold. The incorporation of the differentiation factor into the scaffold may be achieved by standard techniques.

In a preferred embodiment, the differentiation factor is associated with a localized area or region of the scaffold. A particle or bead containing concentrated source of factor is placed onto scaffold and factor is allowed to leach out,

The scaffold may contain 2, 3, 4, 5, or more differentiation factors, each of which may be independently localized to a specific region of the scaffold.

Preferably, the scaffold is sterilized prior to use. PuraMatrix™ and PuraMatrixCST™ may be sterilized using γ-irradiation. Other scaffolds may be sterilized using 70% vol/vol ethanol, followed by washing the scaffold with phosphate buffered saline.

Culturing Homogeneous Multipotent Cells

Following sterilization of the scaffold, this is seeded with at least one homogeneous population of multipotent cells. Typically, this is carried out by simply adding cells suspended in a small volume of culture medium to the scaffold, followed by immersing the scaffold in culture medium. Where the scaffold is PuraMatrix™ and PuraMatrixCST™, or composed of collagen, direct injection of suspended cells in culture medium is also possible.

Where the scaffold is matrigel, cells are suspended in a 50% vol/vol mixture of media and matrigel, which is then allowed to solidify at 37° C., according to methods known in the art. The scaffold is then detached from the culture vessel with a sterile blade.

In one embodiment, a single population of homogeneous multipotent cells is seeded onto the matrix. The invention also encompasses seeding a scaffold with a plurality of populations of homogeneous multipotent cells (or profiles). In a preferred embodiment, 2, 3, 4, 5, 6, 7, 8, 9, 10 or more than 10 populations of homogeneous multipotent cells are seeded onto the scaffold.

As discussed above, the cells of each population of homogeneous multipotent cells are hypothesized to have the same developmental potential, which is distinct from (although possibly overlapping with) the developmental potential of other homogeneous multipotent cells. It is thought that the cells of different populations of homogeneous multipotent cells may interact with one another during the culture process in a manner resulting in the differentiation of one or both populations of cells.

Typically, the cells supported by the scaffold are cultured in the culture medium used to culture the embryonic stem cell line/adult stem cell line from which they were derived. However, the composition of this culture medium may be modified. For example, growth factors or hormones may be added or removed and the pH or nutritional composition of the media may be altered.

In addition, a differentiation factor is present during at least part of the culture process. This differentiation factor may be associated with the scaffold, as mentioned previously. Alternatively, the differentiation factor may be present in the culture medium. In this case, the differentiation factor may be present in only one batch or change of culture medium surrounding the construct.

Each batch of culture medium may contain 2, 3, 4, 5 or more different differentiation factors. The length of exposure of the cells to any particular differentiation factor is easily controlled, by replacing the culture medium after the required time of exposure. Similarly, the timing of exposure can be controlled.

Identification of Differentiated Tissue

Where the initial population of embryonic stem cells or the initial population of adult stem cells contained a reporter gene under the control of a promoter active in one or more differentiated cell types present in the differentiated tissue of interest, production of differentiated tissue is determined by assaying for reporter gene expression, wherein the presence of reporter gene expression is indicative of the presence of the differentiated tissue.

Any method of assaying for reporter gene expression may be used. However, in a preferred embodiment, the reporter gene encodes a fluorescent marker protein and fluorescence confocal microscopy is used to assay reporter gene expression.

Where two or more reporter genes are used, each containing a different fluorescent marker protein under the control of a different tissue specific promoter, it is possible to use fluorescence confocal microscopy to look for more complex patterns of differentiation. For example, it would be possible to screen for “yellow” capillaries (cells expressing eYFP under the control of a capillary specific promoter) surrounded by “green” cardiomyocytes (cells expressing eGFP under the control of a cardiomyocyte specific promoter, such as the myosin heavy chain promoter). In a preferred embodiment, a 2-photon confocal microscope is used since this permits higher order structural features such as tubes, sheets or sinusoids to be identified.

Reporter genes encoding non-fluorescent proteins may also be used. Where these non-fluorescent proteins are recognized by an antibody, the pattern of expression of these reporter genes may be identified using known direct or indirect immunofluorescence methods.

Where the initial population of embryonic stem cells or adult stem cells does not contain a reporter gene, the production of differentiated tissue may be determined by assaying for the presence of markers associated with a differentiated tissue, or for the presence of markers associated with differentiated cell types present in the differentiated tissue, wherein the presence of these markers is indicative of the presence of the differentiated tissue.

The markers may be proteins associated with a differentiated tissue. Surfactant Protein C (SPC) is expressed exclusively in the lung. The presence of marker proteins may be identified using immunohistochemistry methods. For example, the sample may be fixed for 6 h in 10% neutral buffered formalin, processed and embedded in paraffin. The embedded tissue sample may then be sectioned. Sections of the tissue may then be stained using the Biocare Medical Universal HRP-DAB kit (Biocare Medical, Walnut Creek, Calif.) according to the manufacturer's instructions. Alternatively, the sample may be analysed by direct or indirect immunofluorescence, according to known methods.

The markers may also be RNA molecules associated with a differentiated tissue. The pattern of expression of RNA markers may be analyzed by in situ hybridization, according to standard methods.

The methods used to assay for the presence of reporter gene expression, or the presence of protein or RNA tissue specific markers may be used to identify the percentage of multipotent cells which have differentiated. It is preferred that the differentiated tissue obtained contains at least 10% differentiated cells. More preferably, the differentiated tissue contains at least 20%, 30%, 40%, 50%, 60%, 70%, 80% or at least 90% differentiated cells. The percentage of differentiated cells produced may be used to assess the success of the method for producing the differentiated tissue. This permits comparison of different methods, to identify the best available method. In addition, the methods may then be optimized to identify modified methods which result in a greater percentage of differentiated cells.

Use of the Differentiated Tissue

Because the differentiated tissues obtained by the method of the invention express markers characteristic of the native differentiated tissue, it is hypothesized that they will resemble native differentiated tissues in other ways. Consequently, the differentiated tissues obtained by this method are suitable for in vitro studies of that tissue.

In addition, the differentiated tissues may be used in medicine. For example, the differentiated tissue may be used for transplantation or grafting into a patient in need thereof.

Screening Methods

One aspect of the invention is directed to identifying a process for producing a given or specified differentiated tissue. The experimental approach is broadly similar to that described above. However, a plurality of test populations of multipotent cells are processed. A test population is either an isolated population of homogenous multipotent cells, or a mixture of at least two populations of isolated cells. In a preferred embodiment, 2, 3, 4, 5 or more populations of homogeneous multipotent cells are mixed to generate a test population of cells.

Each test population of cells is then seeded onto a solid phase scaffold. In one embodiment, each test population is seeded onto a discrete area of a scaffold. Typically however, separate scaffolds are used for each test population. The test populations are then cultured in the presence or absence of at least one differentiation factor as described above.

In one embodiment, all test populations of cells are exposed to the same culture conditions. In another embodiment, different test populations are exposed to different culture conditions. In yet another embodiment, each test population is cultured in each culture condition used.

The culture conditions may vary in several ways. For example, a different differentiation factor or combination of factors may be used. The timing and duration of exposure to each differentiation factor present may differ as may any other experimental parameter of the test microenvironment. In addition, each differentiation factor may be associated with the scaffold or in the culture medium. Where a differentiation factor is associated with the scaffold, this may be associated with all of the scaffold or in a localized area only.

Mechanical stimulation can be applied to seeded scaffolds by compressing or stretching the scaffolds. Mechanical stress is known to be important for example in bone formation. Cultures can be exposed to gas (air), electrical stimulation, different atmospheric pressures, and different temperatures. Scaffolds may be made of different materials and possess different 3-D structures. Scaffolds may also be coated with matrices that are composed of different materials. These matrices may be added to the scaffold or allowed to form by deposition from resident cells. The liquid phase media may be varied to effect a change in the microenvironment. These include changes in pH, the presence or absence of soluble factors capable of inducing differentiation, proliferation or development, metabolites, small molecules, and dissolved gases.

Each test population may be processed simultaneously, possibly in a separate well of a multiwell plate. Therefore, this screening method has a high throughput.

The cultured cells are then assayed for differentiation as described above. This permits processes for producing a differentiated tissue to be identified. Processes for producing a differentiated tissue are processes (i.e. the combination of test population, culture conditions, scaffolds and additional stimulation) that generate a differentiated cell type present in the differentiated tissue.

The percentage of differentiated cells generated by each process may be determined. In general, the higher the percentage of differentiated cell, the more effective the process for producing a differentiated tissue.

The process for producing a differentiated tissue may be optimized to further increase the percentage of multipotent cells differentiating. This involves modifying at least one parameter of a known process for producing a differentiated tissue to arrive at a modified process, performing the modified process and determining the percentage of differentiated cells generated by the modified method. Where the modified process results in a higher percentage of differentiated cells than the known process, the modified process is an optimized process.

This process may be repeated in an iterative manner to progressively optimize the process. In other words, a parameter of the identified optimized process may be modified and the further modified process can be tested. Where a further improvement in the percentage of differentiated cells is observed, this further modified process is an optimized process.

Experimental parameters are any variable in the process for producing a differentiated tissue and include the identity of the differentiation factor, the concentration of the differentiation factor, the localization of the differentiation factor, the timing and duration of exposure of the multipotent cells to a differentiation factor, and the composition of the population of the test population of multipotent cells and the nature of the scaffold and matrix which covers the scaffold and any additional stimulation (e.g., mechanical, electrical) The “matrix” is a biologically active material in or on the scaffold. A factor is a material which is a soluble material in the culture medium.

Methods are used to understand and model the way 3D microenvironment and its individual components interact and evolve over time. Data collected in this system is expected to be extremely complex and therefore methods will be needed to understand the underlying parameters and principles that determine the outcomes. Rules that will be discovered (two stem cell types together result in one new cell) will be modular knowledge and can be connected with other data sets obtained from these data sets or externally derived data sets.

Data capture: Data are collected from the experimental analyses such as 3D optical data, fluorescence data, structural data, gene expression (transcription or protein expression), a cellular activity, etc.

Data Analysis: Will require sophisticated image processing suite, image processing suite will need to segment images (breaking it into pieces) and look for interesting relationships between the various components of the image. To discover physicologically relevant features. In addition there is segmentation over time. Time series analysis. Pattern recognition is also another important aspect of the data analysis.

Modeling/prediction: The parameters may be modified in a non-random way in accordance with an in silico model of the differentiating tissue. This model could utilize several different computational techniques, for example, cellular automata with autocrine and paracrine (nearest neighbor) rules could be used. Cellular automata performs calculations through interactions of nearest neighbor (cells). This is a simple computational metaphor for the way that components in a system interact with their neighbors. This captures the spatial constraints of the biological system under study.

Alternatively, functional equations for cellular relationships could be established empirically or from first principles (differential equations). The technique that seems likely to produce the best results, though, will be a hybrid of automata (for autocrine and paracrine effects) and functional (endocrine) computing. This accounts for longer range interactions that can be considered to be endocrine in nature. The rules and functions used in these computations can be derived from a combination of expert knowledge and machine learning models trained on experimental data. Modeling controlled development in this fashion will be a computationally intensive process, but the problem can be efficiently parallelized and computation can be multi-threaded. The knowledge gained from these analysis will support the design of experiments that are most likely to yield the desired outcome within a size and scope that is experimentally feasible.

Further details of the proposed screening strategy are given below:

Step I. Stratification of early stage differentiating ESCs into distinct subpopulations: LIF and feeder layers will be withdrawn from ESC cultures to initiate differentiation. Early stage differentiating ESCs will be harvested and screened against a large panel of antibodies to identify those antibodies that can be used to create phenotypic subsets of differentiating cells. A panel of antibodies (profiling antibodies) is first obtained. Antibodies may need to be identified that effectively permit stratification of the heterogeneous population of early stage differentiating cells into defined subpopulations. Subpopulations will thus be defined by the pattern of expression of proteins that are recognized by these profiling antibodies. In addition to the use of profiling antibodies to stratify the cell mixture into subpopulations, ES cell lines may be used that have been engineered to express a reporter gene (such as eGFP) under the control of a lineage and stage specific promoter (e.g., brachyury). Flow cytometry will be used to separate sub populations. Subpopulations may be derived from differentiating ES cell cultures at various timepoints relative to the initiation of differentiation.

Steps I and III. Recombine subpopulations (full factorial design) and seeding of cell combinations onto matrices (±soluble factors): Individual subpopulations can be used alone or in combination which each other to create a large array of unique cellular mixtures. Unary, binary and potentially more complex combinations can be dispensed using sterile automated dispense methods. Cell populations will be directly introduced into sites of an array format such as a microtiter dish or chip that contains defined 3-D matrix. Various parameters of the 3-D scaffold may be tested in this manner including, scaffolding material, matrix coating (e.g., fibronectin), viscoelasticity, and 3-D architecture. In addition to various matrices, it is also possible to test the effect of various media. Media may be supplemented with various growth factors or hormones or they may vary with respect to nutritional composition, metabolites, pH or other stressors. Seeded scaffolds may be subject to additional stimulation including electrical and mechanical.

Step IV. Screen for reporter expression and higher order structure: ESC lines will be used that express fluorescent marker proteins under the control of well defined tissue and stage specific promoters. Such lines can be generated, for example, by homologous recombination (“knock-in” technology). Promoters will be chosen that are expressed in a lineage and stage-specific manner. For example, the fluorescent marker protein that is under the control of a myosin heavy chain promoter will be expressed upon differentiation of ES cells to a cardiomyocyte lineage. Alternatively, expression of the surfactant protein C (SPC) locus is indicative of differentiation towards a lung pathway. Because there are several distinct fluorescent marker proteins (e.g., eGFP, eCFP, eYFP, and DsRed), it is possible to screen for more complex patterns. For example, it might be possible to identify ‘green’ cardiomyocytes surrounded by ‘yellow’ capillaries. Analysis will be carried out using 2-photon confocal microscopy thereby allowing for elucidation of higher order structural features (e.g., tubes, sheets, or sinusoids).

Whilst the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof.

1. A method of screening for a differentiated tissue, which method comprises:
a) obtaining a plurality of samples derived from at least one population of multipotent cells;
b) culturing the cells on a three dimensional scaffold, wherein at least one experimental parameter is varied for each sample; and
c) observing or measuring each sample for controlled development, self-assembly, or differentiation.

2. A method of screening for a differentiated tissue, which method comprises:
a) obtaining a plurality of samples of cells, derived from a population of multipotent cells cultured on a three dimensional scaffold, wherein the cells have undergone controlled development, self-assembly, or differentiation within the three dimensional scaffold;
b) varying at least one experimental parameter for each sample; and
c) observing or measuring each sample for further controlled development, self-assembly, or differentiation.

3. The method of claim 1, wherein the experimental parameter is:
a) an additional cell population;
b) a different scaffold material;
c) a different matrix material;
d) a different factor;
e) amount of time cultured;
f) amount of physical stimulation;
g) temperature;
h) a different scaffold structure;
j) the locating of factors in different positions of a scaffold.

4. The method of claim 2, wherein the experimental parameter is:
a) an additional cell population;
b) a different scaffold material;
c) a different matrix material;
d) a different factor;
e) amount of time cultured;
f) amount of physical stimulation;
g) temperature;
h) a different scaffold structure; or
j) the locating of factors in different positions of a scaffold.

5. The method of claim 1, wherein the cell population is homogeneous for at least one marker.

6. The method of claim 2, wherein the cell population is homogeneous for at least one marker.
